(19) United States
(12) Patent Application Publication
(10) Pub. No.: US 2024/0029881 A1
(43) Pub. Date: Jan. 25, 2024

(54) PLANNING SYSTEM BASED ON RISKS OF SURGICAL ERROR

(71) Applicant: Optum, Inc., Minnetonka, MN (US)

(72) Inventors: Swadeep Singh, Noida (IN); Chandni Nanda, Delhi (IN); Abhay Shukla, Noida (IN)

(21) Appl. No.: 17/812,822

(22) Filed: Jul. 15, 2022

(51) Int. Cl. G16H 50/20; G16H 50/30

(52) U.S. Cl. CPC G16H 50/20; G16H 50/30

(57) ABSTRACT

A method comprises: for each respective factor of a plurality of factors that affect a risk of surgical error occurring when a surgeon performs a surgery on a patient: obtaining, by a computing system, a factor value for the respective factor; and applying, by the computing system, a machine-learned weight for the respective factor to the factor value for the respective factor to determine a weighted factor value for the respective factor; determining a risk score for the surgery based on the weighted factor values for the factors, wherein the risk score is a measure of the risk of surgical error occurring during the surgery given the factor values for the plurality of factors; and generating, by the computing system, based on the risk score for the surgery, a recommendation regarding the surgery.

### TECHNICAL FIELD

This disclosure relates to medical planning systems.

### BACKGROUND

Errors during surgery can lead to serious consequences. For instance, it is estimated that 33% of surgical errors lead to permanent injuries and 6.6% of surgical errors lead to wrongful deaths. There are many factors that increase the likelihood of surgical errors. For example, deficiencies in education, training, and experience are associated with increased likelihoods of surgical errors. Fatigue, inadequate staffing, and poor supervision are also associated with increased likelihoods of surgical errors.

### SUMMARY

This disclosure describes computer-implemented techniques that use machine learning to make recommendations regarding scheduling surgeries, such as whether to assign individual surgeons or surgical teams to individual surgeries, as one example, in order to reduce risks of surgical errors. As described in this disclosure, for each respective factor of a plurality of factors that affect a risk of surgical error occurring when a surgeon performs a surgery, a computing system may obtain a factor value for the respective factor. Additionally, the computing system may apply a machine-learned weight for the respective factor to the factor value for the respective factor to determine a weighted factor value for the respective factor. The computing system may determine a risk score for the surgery based on the weighted factor values for the factors. The risk score may be a measure of the risk of surgical error occurring while a specific set of factor values apply with respect to the surgery. The computing system may generate, based on the risk score for the surgery, a recommendation regarding scheduling the surgery, such as whether to assign the surgeon to the surgery, whether to assign the surgeon to the surgery with a different support team, whether to assign a particular facility or operating theater for the surgery, and so on.

In one aspect, this disclosure describes a method comprising: for each respective factor of a plurality of factors that affect a risk of surgical error occurring when a surgeon performs a surgery on a patient: obtaining, by a computing system, a factor value for the respective factor; and applying, by the computing system, a machine-learned weight for the respective factor to the factor value for the respective factor to determine a weighted factor value for the respective factor; determining, by the computing system, a risk score for the surgery based on the weighted factor values for the factors, wherein the risk score is a measure of the risk of surgical error occurring during the surgery given the factor values for the plurality of factors; and generating, by the computing system, based on the risk score for the surgery, a recommendation regarding scheduling the surgery.

In another example, this disclosure describes a computing system comprising: one or more storage devices configured to store factor values for a plurality of factors that affect a risk of surgical error occurring when a surgeon performs a surgery on a patient; and processing circuitry configured to: for each respective factor of the plurality of factors: obtain a factor value for the respective factor; and apply a machine-learned weight for the respective factor to the factor value for the respective factor to determine a weighted factor value for the respective factor; determine a risk score for the surgery based on the weighted factor values for the factors, wherein the risk score is a measure of the risk of surgical error occurring during the surgery given the factor values for the plurality of factors; and generate, based on the risk score for the surgery, a recommendation regarding scheduling the surgery.

In another example, this disclosure describes a non-transitory computer-readable storage medium having instructions stored thereon that, when executed, cause a computing system to: for each respective factor of a plurality of factors that affect a risk of surgical error occurring when a surgeon performs a surgery on a patient: obtain a factor value for the respective factor; and apply a machine-learned weight for the respective factor to the factor value for the respective factor to determine a weighted factor value for the respective factor; determine a risk score for the surgery based on the weighted factor values for the factors, wherein the risk score is a measure of the risk of surgical error occurring during the surgery given the factor values for the plurality of factors; and generate, based on the risk score for the surgery, a recommendation regarding scheduling the surgery.

In another example, this disclosure describes a non-transitory computer-readable storage medium having instructions stored thereon that, when executed, cause processing circuitry to:

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF DRAWINGS

FIG. 1 is a block diagram illustrating an example computing system in accordance with one or more aspects of this disclosure.

FIG. 2 is a flowchart illustrating an example operation of the computing system, in accordance with one or more aspects of this disclosure.

FIG. 3 is a flowchart illustrating an example operation of the computing system to determine a weighted factor value for a factor, in accordance with one or more aspects of this disclosure.

FIG. 4 is a conceptual diagram illustrating an example of logistic regression, in accordance with one or more aspects of this disclosure.

### DETAILED DESCRIPTION

Surgical errors may have serious consequences, including permanent injury and wrongful death. Example types of surgical errors may include operating on the wrong site, performing the wrong procedure, perform the surgery on the wrong patient, omitting steps of surgical procedures, leaving surgical items in patients, anesthesia injuries, and other errors that lead to adverse outcomes.

This disclosure describes a computing system configured to apply machine learning (ML) techniques to reduce the likelihood of surgical errors, such as by providing recommendations regarding whether specific surgeons should be assigned to specific surgeries, as one example. For instance, a recommendation may indicate that surgeon A should not be assigned to surgery B because there may be an increased likelihood of surgical error if surgeon A performs surgery B. The example techniques may also be used in other ways to reduced likelihood of surgical errors, and providing recommendations regarding whether specific surgeons should be assigned to specific surgeries is one example.

As described herein, for each respective factor of a plurality of factors that affects a risk of surgical error occurring when a surgeon performs a surgery, a computing system may obtain a factor value for the respective factor. Additionally, the computing system may apply a machine-learned weight for the respective factor to the factor value for the respective factor to determine a weighted factor value for the respective factor. The computing system may determine a risk score for the surgery based on the weighted factor values for the factors. The risk score is a measure of the risk of surgical error occurring during the surgery given the factor values for the plurality of factors. The computing system may generate, based on the risk score for the surgery, a recommendation regarding the surgery, such as whether to assign the surgeon to the surgery. The use of machine-learned weights may enhance the accuracy of the computing system and may ensure that the factors are given weights that appropriately reflect the importance of factors in predicting the risk of surgical error.

In some examples, the computing system may learn the machine-learned weights using logistic regression. Applying a machine-learning process that uses logistic regression to determine the weights may require relatively low computation power compared with other machine learning models. Additionally, the chances of overfitting are low when using logistic regression. Furthermore, logistic regression is typically used to make a binary prediction for one or more input values. In some examples of this disclosure, the weight may be a coefficient that defines a shape of a logistic regression curve as a whole, not a binary prediction made using the logistic regression curve at a specific input value.

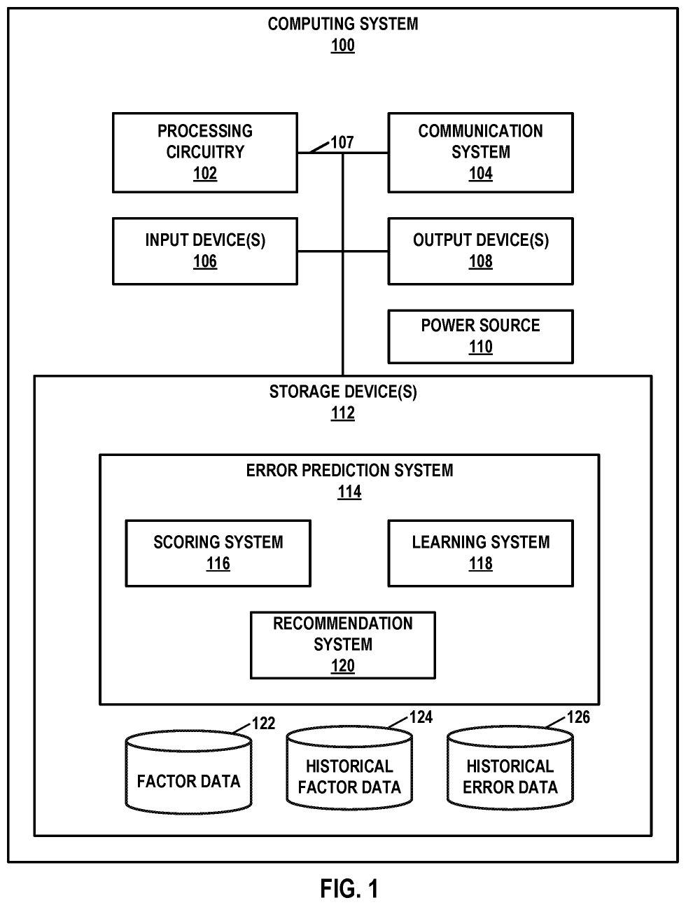

FIG. 1 is a block diagram illustrating an example computing system 100 in accordance with one or more aspects of this disclosure. FIG. 1 illustrates only one particular example of computing system 100, and many other example configurations of computing system 100 exist. Computing system 100 may comprise one or more computing devices. Example types of computing devices may include personal computers, server computers, smartphones, laptop computers, tablet computers, and so on.

As shown in the example of FIG. 1, computing system 100 includes processing circuitry 102, a communication system 104, one or more input devices 106, one or more output device(s) 108, a power source 110, one or more storage device(s) 112, and one or more communication channels 107. Computing system 100 may include other components. For example, computing system 100 may include physical buttons, microphones, speakers, communication ports, and so on. Communication channel(s) 107 may interconnect each of processing circuitry 102, communication system 104, input device(s) 106, output device(s) 108, and storage device(s) 112 for inter-component communications (physically, communicatively, and/or operatively). In some examples, communication channel(s) 107 may include a system bus, a network connection, an inter-process communication data structure, or any other method for communicating data. Power source 110 may provide electrical energy to processing circuitry 102, communication system 104, input device(s) 106, output device(s) 108, and storage device(s) 112.

Processing circuitry 102 may include circuits configured to perform various processing activities. Processing circuitry 102 may include microprocessors, application-specific integrated circuits, field-programmable gate arrays (FPGAs), and so on. Processing circuitry 102 may be distributed among one or more computing devices of computing system 100.

Storage device(s) 112 may store information required for use during operation of computing system 100. In some examples, storage device(s) 112 have the primary purpose of being a short-term and not a long-term computer-readable storage medium. Storage device(s) 112 may include volatile memory and may therefore not retain stored contents if powered off. In some examples, storage device(s) 112 includes non-volatile memory that is configured for long-term storage of information and for retaining information after power on/off cycles. In some examples, processing circuitry 102 of computing system 100 may read and execute instructions stored by storage device(s) 112.

Computing system 100 may include one or more input devices 106 that computing system 100 uses to receive user input. Examples of user input include tactile, audio, and video user input. Input device(s) 106 may include presence-sensitive screens, touch-sensitive screens, mice, keyboards, voice responsive systems, microphones, motion sensors capable of detecting gestures, or other types of devices for detecting input from a human or machine.

Communication system 104 may enable computing system 100 to send data to and receive data from one or more other computing devices (e.g., via a communication network, such as a local area network or the Internet). In some examples, communication system 104 may include wireless transmitters and receivers that enable computing system 100 to communicate wirelessly with the other computing devices. Examples of communication system 104 may include network interface cards, Ethernet cards, optical transceivers, radio frequency transceivers, or other types of devices that are able to send and receive information. Other examples of such communication units may include BLUETOOTH™, 3G, 4G, 5G, and WI-FI™ radios, Universal Serial Bus (USB) interfaces, etc.

Output device(s) 108 may generate output. Examples of output include tactile, audio, and video output. Output device(s) 108 may include presence-sensitive screens, sound cards, video graphics adapter cards, speakers, liquid crystal displays (LCD), light emitting diode (LED) displays, or other types of devices for generating output. In some examples, output device(s) 108 may include virtual reality, augmented reality, or mixed reality display devices.

Processing circuitry 102 may read instructions from storage device(s) 112 and may execute instructions stored by storage device(s) 112. Execution of the instructions by processing circuitry 102 may configure or cause computing system 100 to provide at least some of the functionality ascribed in this disclosure to computing system 100 or components thereof (e.g., processing circuitry 102). In the example of FIG. 1, storage device(s) 112 include computer-readable instructions associated with an error prediction system 114. Error prediction system 114 is configured to determine a risk score for a surgery. The risk score for the surgery is a measure of a risk of surgical error occurring during the surgery given factor values of the plurality of factors apply with respect to the surgery. Such factor values may indicate the skill level of a surgeon, information regarding utilization of the surgeon, information about a surgical support team, information about a surgical facility, information about the patient, and so on. Error prediction system 114 may also generate, based on the risk score, a recommendation regarding the surgery, such as whether to assign the specific surgeon to the surgery.

In the example of FIG. 1, error prediction system 114 includes a scoring system 116, a learning system 118, and a recommendation system 120. Scoring system 116 is configured to determine a risk score for a surgery. As part of determining the risk score for the surgery, scoring system 116 may, for each respective factor of a plurality of factors that affect a risk of surgical error occurring when a surgeon performs the surgery, obtain a factor value for the respect factor. Scoring system 116 may determine the factor values based on factor data 122. Factor data 122 may include data from one or more data sources, such as health insurance claim databases, hospital administration databases, patient health record databases, and so on.

Example factors may include one or more of years of experience of the surgeon, average number of patients treated by the surgeon per time period, average number of surgeries performed by the surgeon per time period, utilization percentage of the surgeon, burnout level of the surgeon, patient panel burden of the surgeon, experience level of an anesthesiologist, quality of the anesthesiologist, rating of operating room, experience level of nurse, patient allergies, patient historical surgeries, patient comorbidities, and so on.

Additionally, scoring system 116 may apply a machine-learned weight for the respective factor to the factor value for the respective factor to determine a weighted factor value for the respective factor. The value of the machine-learned weight for the respective factor may correspond to the importance that the respective factor has relative to other factors. For example, a first factor has a relatively low importance, but a second factor has a relatively high importance. Therefore, the machine-learned weight for first factor may indicate relatively low importance, and machine-learned weight for the second factor may indicate relatively high importance. The machine learning techniques may determine which factors are of relatively low importance, and which factors are of relatively high importance.

Learning system 118 is configured to perform one or more machine learning (ML) processes to determine the weights that scoring system 116 applies to the factor values to determine the weighted factor values. For instance, for one or more of the factors, learning system 118 may obtain historical factor data 124 that comprises records of factor values of the factor for a plurality of historical surgeries. Learning system 118 may also obtain historical error data 126 comprising records of whether surgical error occurred during the historical surgeries or other data that may serve as a proxy for the occurrence of surgical error, such as death, readmission, units of blood required, and so on. Learning system 118 may perform a logistic regression on historical factor data 124 and historical error data 126 to determine a logistic regression curve. Learning system 118 may determine the weight for the factor based on a coefficient that defines the logistic regression curve. In some examples, the weight for the factor is equal to the coefficient or is calculated from the coefficient. In some examples, for each respective factor of a plurality of factors, the machine-learned weight for the respective factor is a respective coefficient of the logistic regression curve. In other examples, learning system 118 may use other machine learning models to determine weights based on historical factor data 124 and historical error data 126. For example, learning system 118 may use a support vector machine, an XGBoost model, a naïve Bayes model, or another machine learning model to determine the weights for the factors.

In some examples, learning system 118 may use an ensemble learning model that performs an iterative process that trains multiple ML models using different sets of hyperparameters. Each of the ML models may take factor values (or weighted factor values) as input. Furthermore, in such examples, learning system 118 may determine weights for the ML models using an optimization process. Subsequently, recommendation system 118 may apply the weights to predictions generated by models to determine a risk score for the surgery.

Learning system 118 may update the records (e.g., historical factor data 124) with factor values of the factors that affect the risk of surgical error occurring when the surgeon performs the surgery on the patient. Learning system 118 may update the second records (historical error data 126) to indicate whether surgical error occurred when the surgeon performed the surgery on the patient. Learning system 118 may update the weights for the factors based on these records. Thus, learning system 118 may continue to refine the weights for the factors.

Recommendation system 120 may generate, based on the risk score for the surgery, a recommendation regarding the surgery. For example, recommendation system 120 may generate a recommendation indicating that the surgeon should not be assigned to the surgery if the risk score for the surgery is above a threshold. Conversely, recommendation system 120 may generate a recommendation indicating that the surgeon should be assigned to the surgery if the risk score for the surgery is below the threshold. In some examples, recommendation system 120 may cause one or more of output device(s) 108 to output the recommendation for display to a user of computing system 100. The user of computing system 100 may be a medical scheduler, hospital administrator, surgeon, nurse, or other type of person. In some examples, scoring system 116 may determine risk scores for the surgery for multiple different surgeons. Recommendation system 120 may rank the surgeons. Recommendation system 120 may determine whether to assign a surgeon to the surgery based on the ranking of surgeons. For instance, recommendation system 120 may generate a recommendation to assign a surgeon to the surgery if the surgeon is the top ranked surgeon.

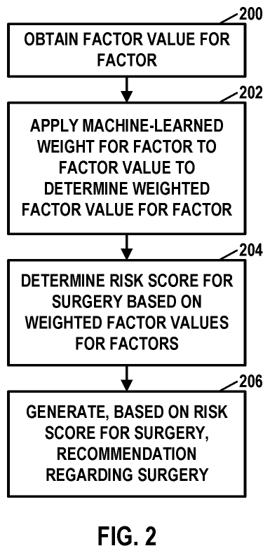

FIG. 2 is a flowchart illustrating an example operation of computing system 100, in accordance with one or more aspects of this disclosure. The flowcharts of this disclosure are provided as examples. Other example operations in accordance with the techniques of this disclosure may include more, fewer, or different actions, or actions in different orders.

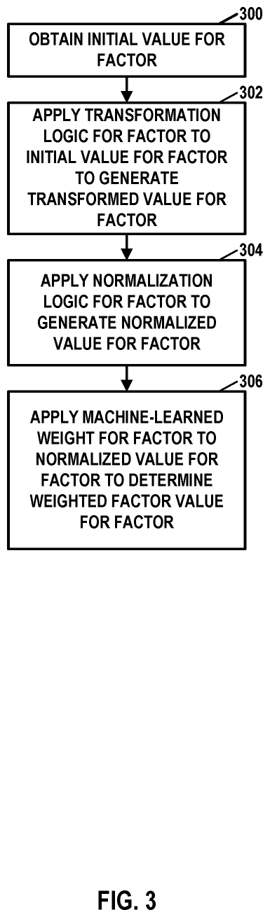

In the example of FIG. 2, scoring system 116 of computing system 100 may obtain a factor value for a factor (200). The factor value for the factor is a numerical value. Additionally, scoring system 116 may apply a machine-learned weight for the factor to the factor value for the factor to determine a weighted factor value for the factor (202). FIG. 3, which is described in greater detail elsewhere in this disclosure, describes an example operation for obtaining a factor value for a factor and applying the machine-learned weight for the factor to determine the weighted factor value for the factor. Scoring system 116 may repeat steps 200 and 202 for each respective factor of a plurality of factors that affect a risk of surgical error occurring when the surgeon performs the surgery on the patient.

Furthermore, in the example of FIG. 2, scoring system 116 may determine a risk score for the surgery based on the weighted factor values for the factors (204). The risk score may be a measure of the risk of surgical error occurring during the surgery given the factor values of the factors. In some examples, scoring system 116 may determine the risk score for the surgery by summing the weighted factor values for the factors to determine an initial risk score for the surgery and normalizing the initial risk score for the surgery to determine the risk score for the surgery. In some examples, scoring system 116 may normalize the initial risk score for the surgery by converting the initial risk score for the surgery to a value on a scale of 0 to 100.

Recommendation system 120 may generate, based on the risk score for the surgery, a recommendation regarding scheduling the surgery (206). For example, recommendation system 120 may generate a recommendation to assign the surgeon to the surgery when the risk score for the surgery is less than a threshold and may generate a recommendation not to assign the surgeon to the surgery when the risk score is greater than the threshold. In some examples, a patient prefers a particular surgeon. In such examples, recommendation system 120 may rerun the process of FIG. 2 multiple times for different time slots for the particular surgeon to obtain risk scores for the surgery for the different time slots for the particular surgeon. Because the particular surgeon may experience different amounts of fatigue in the different time slots, rerunning the process of FIG. 2 for different time slots for the particular surgeon may result in different risk scores. Recommendation system 120 may then generate a recommendation on which of the time slots to schedule the particular surgeon to perform the surgery on the patient.

FIG. 3 is a flowchart illustrating an example operation of computing system 100 to determine a weighted factor value for a factor, in accordance with one or more aspects of this disclosure. In the example of FIG. 3, scoring system 116 of computing system 100 may obtain an initial value for a factor (300). For instance, scoring system 116 may obtain the initial value from a database storing information about the surgeon. Scoring system 116 may obtain initial values for factors from one or more data sources. For example, the factors may include a factor that indicates the number of times the surgeon has performed the surgery. In this example, scoring system 116 may obtain the initial value for this factor from a database containing records of the surgeries performed by the surgeon. In another example, the factors may include a factor that indicates whether the patient has a specific comorbidity. In this example, scoring system 116 may obtain the initial value for the factor from diagnostic codes recorded in a database that stores health insurance claims.

In some examples, scoring system 116 performs a process that generates the initial value from multiple pieces of data and/or multiple data sources. For example, the factors may include a surgeon burnout index. The surgeon burnout index is a measure of mental exhaustion of the surgeon. In this example, scoring system 116 may determine the surgeon burnout index based on several pieces of information, such as the surgeon's length of shifts, numbers of surgeries performed in a given time period preceding the surgery, biometric information regarding the surgeon (e.g., sleep duration, sleep schedule regularity, heart rate patterns, exercise habits, etc.), and other types of information. In this example, scoring system 116 may apply a formula, such as weighted sum, that generates the surgeon burnout index based on the data.

In some examples, scoring system 116 may apply a machine learning process to determine an initial value for a factor based on a plurality of pieces of data. For instance, scoring system 116 may apply a k-means clustering process that identifies clusters of data values associated with different initial values of a factor. In another example, scoring system 116 may apply a neural network model that outputs an initial value of a factor.

Furthermore, in the example of FIG. 3, scoring system 116 may apply transformation logic for the factor to the initial value for the factor to generate a transformed value for the factor (302). The transformation logic for a factor is a process for transforming the initial value for the factor to a different form. There may be different transformation logic for different factors. In other words, the transformation logic for one factor may be different from transformation for another factor of the plurality of factors. Moreover, for some factors, scoring system 116 does not apply transformation logic to the initial values of the factors, meaning that the initial values for the factors are the same as the transformed values for the factors.

As an example of transformation logic, the factors may include an operating room factor. The operating room factor indicates a quality of the operating room (e.g., in terms of equipment, proximity to resources, size, etc.). In this example, the initial value of the operating room factor may indicate which operating room is scheduled to be used to perform the surgery. In this example, the transformation logic for the operating room factor may change the initial value of the operating room factor to a numerical value. In this example, the transformation logic may use a predefined mapping of operating room identifiers to numerical values to transform an operating room identifier to a numerical value. In another example of transformation logic, the transformation logic may convert data indicating counts of deaths and readmissions in particular operating rooms to numerical values for the operating rooms. In another example of transformation logic, the transformation logic may convert an average of key equipment in an operation room into a numerical value for the operating room. In another example, the factors may include a team efficiency factor. In this example, the initial value of the surgical team factor may be an identifier of a surgical team. The transformation logic for the team efficiency factor may transform the identifier of the surgical team to a numerical value indicating a total numbers of years of work experience in the surgical team.

After generating the transformed value for the factor, scoring system 116 may apply a normalization process for the factor to the transformed value for the factor to generate a normalized value for the factor (304). The normalized value for the factor may be the “factor value for the factor” discussed elsewhere in this disclosure. The normalization process for a factor may normalize the transformed value of the factor so that the resulting normalized value for the factor is within the same range as normalized values for other factors. For instance, the normalization process for each of the factors may normalize the transformed values of the factors to a range of 0 to 1. In some examples, the normalization process may be referred to as a scaling process that scales the transformed value to a value in a range of a scale.

There may be different normalization process for different factors. In other words, the normalization process for one factor may be different from a normalization process for the other factor. For example, the normalization process for some factors may be to determine a percentile of the transformed value. For instance, one of the factors may be the average number of patients handled by the surgeon. In this instance, scoring system 116 may compare the average number of patients handled by the surgeon to the average number of patients handled by other surgeons to determine the percentile of the surgeon relative to the other surgeons. Scoring system 116 may then use the percentile as the normalized value for the factor. In other examples, the normalization processes for factors may include scaling to a range, clipping, log scaling, z-score, and so on. In some examples, scoring system 116 does not perform any normalization process on the transformed values of one or more of the factors.

In some examples, scoring system 116 may apply 1-Hot encoding to the normalized value for the factor. 1-Hot encoding changes the format of the normalized value to a format in which only 1 bit of a code has a 1 value and the rest of the code have 0 values. The use of 1-Hot encoding may enable the normalized factor to be used as input to specific types of machine learning models, such as neural networks and clustering models.

Scoring system 116 may apply a machine-learned weight for the factor to the normalized value for the factor to determine a weighted factor value for the factor (306). In some examples, scoring system 116 may multiply the machine-learned weight for the factor and the normalized value for the factor to determine the weighted factor value for the factor.

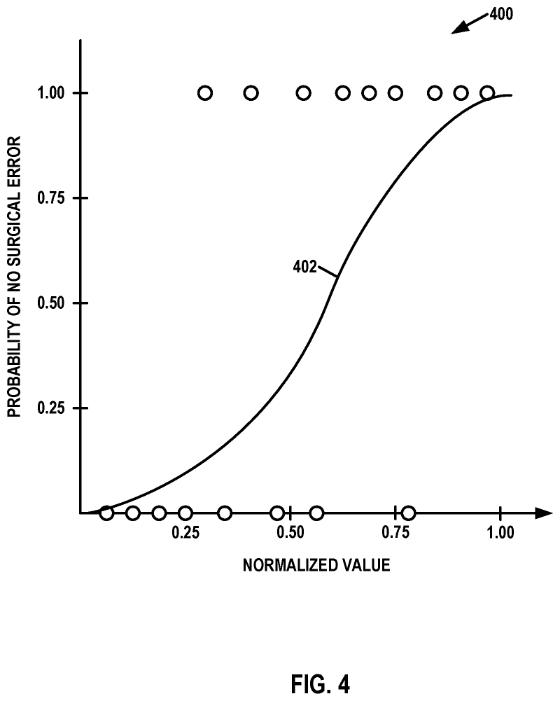

FIG. 4 is a conceptual diagram illustrating an example of logistic regression, in accordance with one or more aspects of this disclosure. Learning system 118 may perform a logistic regression on historical factor data 124 and historical error data 126 to determine a logistic regression curve for one or more factors. In the example of FIG. 4, normalized values of a factor are shown on the horizontal axis of a graph 400 and probabilities of no surgical error occurring are shown on a vertical axis of graph 400. In examples where the logistic curve is for more than one factor, there may be an axis for each of the factors. Circles in graph 400 correspond to individual surgeries and indicate whether or not a surgical error occurred during the corresponding surgery. As shown in the example of FIG. 4, surgical error is more likely to occur when the normalized value of the factor is low and less likely to occur when the normalized value of the factor is high.

Conceptually, learning system 118 may use positions of the circles to apply logistic regression that determines a curve 402 for the factor. Curve 402 is a sigmoid curve that may be defined by the logistic function:

$$p ( x ) = \frac{1}{1 + {e}^{- ( {β}_{0} + {β}_{1} {x}_{1} + … + {β}_{n} {x}_{n} )}} \qquad ( 1 )$$

In equation 1, β₀ is a y-intercept value. In some examples of this disclosure, β₀ is omitted or assumed to be 0. x₁ to xₙ are factor values for respective factors 1 to n, where n is an integer value indicating the number of factors. β₁ . . . βₙ are weights for the factors. The machine learning process determines (i.e., learns) values of β₀ (or β₁) to βₙ that provide a best fit for the data.

Learning system 118 may perform an iterative process to determine the values of β. Learning system 118 may initialize the values of β to be random values. The iterative process that begins with a tentative solution (a curve defined by equation 1) and revises values of β of the tentative solution until no additional improvement is obtained. During each iteration of the iterative process, learning system 118 may compute a loss value. In some examples, learning system 118 may compute the loss value as shown in equation 2, below:

$$ℓ = {\sum }_{k = 1}^{K} ( {y}_{k} ln ( {p}_{k} ) + ( 1 - {y}_{k} ) ln ( 1 - {p}_{k} ) ) \qquad ( 2 )$$

In equation 2, l is the loss value, yₖ is the y-axis value of the k-th data point (e.g., the k-th circle of FIG. 4); ln( ) is the natural log function; and pₖ is a probability predicted using the current tentative solution. pₖ may be calculated as shown above in equation (1), where the k-th data point is a vector of values x₁ . . . xₙ. When using minimum likelihood estimation, learning system 118 may attempt to maximize the loss value. The loss value may be minimize when derivatives of the loss value with respect to each of the β values is equal to 0. In this example, the partial derivatives of the loss function with respect to βⱼ (where j is a value from 0 to n) may be defined as:

$$\frac{\partial ℓ}{\partial {β}_{j}} = \frac{1}{m} {\sum }_{k = 1}^{m} ( σ ( {β}^{T} \cdot {x}^{( k )} ) - {y}^{( k )} ) {x}_{j}^{( k )} \qquad ( 3 )$$

In equation (3), βⱼ is a coefficient, β^{T} is a transposed vector of the β values, x^{(k)} is the vector of values of the k-th data point, σ(β^{T}·x^{(k)}) is the probability predicted by the tentative solution for the k-th data point, m is the number of data points, y^{(k)} is y-axis value of the k-th data point, xⱼ^{(k)} is the value of the j-th value of the k-th data point. The value of xⱼ^{(k)} for j=0 is assumed to be equal to 1. The set of partial derivatives of the loss function with respect to β₀ to βₙ may be referred to as a gradient vector (gradients).

Learning system 118 may determine an updated vector of β values (i.e., β) as follows:

β=β−η*gradients (4)

In equation (4), η is a learning rate, β is the vector of β values, and gradients is the gradient vector. Learning system 118 may then use the β values as the basis for another tentative solution and repeat the process above. Learning system 118 may continue performing iterations in this way until a termination criterion is reached, such as a fixed number of iterations, until the total of changes in vector of β are below a specific threshold, or other conditions at which training may be considered complete. After training is complete, scoring system 116 may use the values of β₁ . . . βₙ as the weights for factors. In this way, scoring system 116 may use coefficients (i.e., values of β₁ . . . βₙ) that define a logistic regression curve as weights for factors that affect the risk of surgical error occurring when a surgeon performs a surgery on a patient.

The following paragraphs provide a non-limiting list of examples in accordance with techniques of this disclosure.

Example 1: A method includes for each respective factor of a plurality of factors that affect a risk of surgical error occurring when a surgeon performs a surgery on a patient: obtaining, by a computing system, a factor value for the respective factor; and applying, by the computing system, a machine-learned weight for the respective factor to the factor value for the respective factor to determine a weighted factor value for the respective factor; determining, by the computing system, a risk score for the surgery based on the weighted factor values for the factors, wherein the risk score is a measure of the risk of surgical error occurring during the surgery given the factor values for the plurality of factors; and generating, by the computing system, based on the risk score for the surgery, a recommendation regarding scheduling the surgery.

Example 2: The method of example 1, wherein the method further comprises, for at least one factor of the plurality of factors: obtaining, by the computing system, first records of factor values of the factor for a plurality of historical surgeries and second records of whether surgical error occurred during the historical surgeries; performing, by the computing system, a logistic regression on the first records and the second records to determine a logistic regression curve; and determining, by the computing system, the weight for the factor is a coefficient that defines the logistic regression curve.

Example 3: The method of example 2, wherein, for each respective factor of the plurality of factors, the machine-learned weight for the respective factor is a respective coefficient of the logistic regression curve.

Example 4: The method of any of examples 2 and 3, further includes updating the first records with factor values of the factors that affect the risk of surgical error occurring when the surgeon performs the surgery on the patient; and updating the second records to indicate whether surgical error occurred when the surgeon performed the surgery on the patient.

Example 5: The method of any of examples 1 through 4, wherein obtaining the factor value for the respective factor comprises: obtaining, by the computing system, an initial value for the respective factor; applying, by the computing system, transformation logic for the respective factor to the initial value for the respective factor to determine a transformed value for the respective factor; and applying, by the computing system, a normalization process for the respective factor to the transformed value for the respective factor to determine the factor value for the respective value.

Example 6: The method of example 5, wherein at least one of: the transformation logic for the respective factor is different from transformation for another factor of the plurality of factors, or the normalization process for the respective factor is different from a normalization process for the other factor.

Example 7: The method of any of examples 1 through 6, wherein determining the risk score for the surgery based on the weighted factor values for the factors comprises: summing, by the computing system, the weighted factor values for the factors to determine an initial risk score for the surgery; and normalizing, by the computing system, the initial risk score for the surgery to determine the risk score for the surgery.

Example 8: The method of any of examples 1 through 7, wherein the factors include one or more of: years of experience of the surgeon, average number of patients treated by the surgeon per time period, average number of surgeries performed by the surgeon per time period, utilization percentage of the surgeon, burnout level of the surgeon, patient panel burden of the surgeon, experience level of an anesthesiologist, quality of the anesthesiologist, rating of operating room, experience level of nurse, patient allergies, patient historical surgeries, or patient comorbidities.

Example 9: A computing system includes one or more storage devices configured to store factor values for a plurality of factors that affect a risk of surgical error occurring when a surgeon performs a surgery on a patient; and processing circuitry configured to: for each respective factor of the plurality of factors: obtain a factor value for the respective factor; and apply a machine-learned weight for the respective factor to the factor value for the respective factor to determine a weighted factor value for the respective factor; determine a risk score for the surgery based on the weighted factor values for the factors, wherein the risk score is a measure of the risk of surgical error occurring during the surgery given the factor values for the plurality of factors; and generate, based on the risk score for the surgery, a recommendation regarding scheduling the surgery.

Example 10: The computing system of example 9, wherein the processing circuitry is further configured to, for at least one factor of the plurality of factors: obtain first records of factor values of the factor for a plurality of historical surgeries and second records of whether surgical error occurred during the historical surgeries; perform a logistic regression on the first records and the second records to determine a logistic regression curve; and determine the weight for the factor is a coefficient that defines the logistic regression curve.

Example 11: The computing system of example 10, wherein, for each respective factor of the plurality of factors, the machine-learned weight for the respective factor is a respective coefficient of the logistic regression curve.

Example 12: The computing system of any of examples 10 and 11, wherein the processing circuitry is further configured to: update the first records with factor values of the factors that affect the risk of surgical error occurring when the surgeon performs the surgery on the patient; and update the second records to indicate whether surgical error occurred when the surgeon performed the surgery on the patient.

Example 13: The computing system of any of examples 9 through 12, wherein the processing circuitry is configured to, as part of obtaining the factor value for the respective factor: obtain an initial value for the respective factor; apply transformation logic for the respective factor to the initial value for the respective factor to determine a transformed value for the respective factor; and apply a normalization process for the respective factor to the transformed value for the respective factor to determine the factor value for the respective value.

Example 14: The computing system of example 13, wherein at least one of: the transformation logic for the respective factor is different from transformation for another factor of the plurality of factors, or the normalization process for the respective factor is different from a normalization process for the other factor.

Example 15: The computing system of any of examples 9 through 14, wherein the processing circuitry is configured to, as part of determining the risk score for the surgery based on the weighted factor values for the factors: sum the weighted factor values for the factors to determine an initial risk score for the surgery; and normalize the initial risk score for the surgery to determine the risk score for the surgery.

Example 16: The computing system of any of examples 9 through 15, wherein the factors include one or more of: years of experience of the surgeon, average number of patients treated by the surgeon per time period, average number of surgeries performed by the surgeon per time period, utilization percentage of the surgeon, burnout level of the surgeon, patient panel burden of the surgeon, experience level of an anesthesiologist, quality of the anesthesiologist, rating of operating room, experience level of nurse, patient allergies, patient historical surgeries, or patient comorbidities.

Example 17: A non-transitory computer-readable storage medium having instructions stored thereon that, when executed, cause a computing system to: for each respective factor of a plurality of factors that affect a risk of surgical error occurring when a surgeon performs a surgery on a patient: obtain a factor value for the respective factor; and apply a machine-learned weight for the respective factor to the factor value for the respective factor to determine a weighted factor value for the respective factor; determine a risk score for the surgery based on the weighted factor values for the factors, wherein the risk score is a measure of the risk of surgical error occurring during the surgery given the factor values for the plurality of factors; and generate, based on the risk score for the surgery, a recommendation regarding scheduling the surgery.

Example 18: The non-transitory computer-readable storage medium of example 17, wherein the instructions, when executed further cause the computing system to, for at least one factor of the plurality of factors: obtain first records of factor values of the factor for a plurality of historical surgeries and second records of whether surgical error occurred during the historical surgeries; perform a logistic regression on the first records and the second records to determine a logistic regression curve; and determine the weight for the factor is a coefficient that defines the logistic regression curve.

Example 19: The non-transitory computer-readable storage medium of example 18, wherein, for each respective factor of the plurality of factors, the machine-learned weight for the respective factor is a respective coefficient of the logistic regression curve.

Example 20: The non-transitory computer-readable storage medium of any of examples 17 through 19, wherein the instructions that cause the computing system to obtain the factor value for the respective factor comprise instructions that, when executed, cause the computing system to: obtain an initial value for the respective factor; apply transformation logic for the respective factor to the initial value for the respective factor to determine a transformed value for the respective factor; and apply a normalization process for the respective factor to the transformed value for the respective factor to determine the factor value for the respective value.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processing circuits to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, cache memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Functionality described in this disclosure may be performed by fixed function and/or programmable processing circuitry. For instance, instructions may be executed by fixed function and/or programmable processing circuitry. Such processing circuitry may include one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. Processing circuits may be coupled to other components in various ways. For example, a processing circuit may be coupled to other components via an internal device interconnect, a wired or wireless network connection, or another communication medium.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples have been described. These and other examples are within the scope of the following claims.

What is claimed is:

1. A method comprising:
for each respective factor of a plurality of factors that affect a risk of surgical error occurring when a surgeon performs a surgery on a patient:
obtaining, by a computing system, a factor value for the respective factor; and
applying, by the computing system, a machine-learned weight for the respective factor to the factor value for the respective factor to determine a weighted factor value for the respective factor;
determining, by the computing system, a risk score for the surgery based on the weighted factor values for the factors, wherein the risk score is a measure of the risk of surgical error occurring during the surgery given the factor values for the plurality of factors; and
generating, by the computing system, based on the risk score for the surgery, a recommendation regarding scheduling the surgery.

2. The method of claim 1, wherein the method further comprises, for at least one factor of the plurality of factors:
obtaining, by the computing system, first records of factor values of the factor for a plurality of historical surgeries and second records of whether surgical error occurred during the historical surgeries;
performing, by the computing system, a logistic regression on the first records and the second records to determine a logistic regression curve; and
determining, by the computing system, the weight for the factor is a coefficient that defines the logistic regression curve.

3. The method of claim 2, wherein, for each respective factor of the plurality of factors, the machine-learned weight for the respective factor is a respective coefficient of the logistic regression curve.

4. The method of claim 2, further comprising:
updating the first records with factor values of the factors that affect the risk of surgical error occurring when the surgeon performs the surgery on the patient; and
updating the second records to indicate whether surgical error occurred when the surgeon performed the surgery on the patient.

5. The method of claim 1, wherein obtaining the factor value for the respective factor comprises:
obtaining, by the computing system, an initial value for the respective factor;
applying, by the computing system, transformation logic for the respective factor to the initial value for the respective factor to determine a transformed value for the respective factor; and
applying, by the computing system, a normalization process for the respective factor to the transformed value for the respective factor to determine the factor value for the respective value.

6. The method of claim 5, wherein at least one of:
the transformation logic for the respective factor is different from transformation for another factor of the plurality of factors, or
the normalization process for the respective factor is different from a normalization process for the other factor.

7. The method of claim 1, wherein determining the risk score for the surgery based on the weighted factor values for the factors comprises:
summing, by the computing system, the weighted factor values for the factors to determine an initial risk score for the surgery; and
normalizing, by the computing system, the initial risk score for the surgery to determine the risk score for the surgery.

8. The method of claim 1, wherein the factors include one or more of:
years of experience of the surgeon,
average number of patients treated by the surgeon per time period,
average number of surgeries performed by the surgeon per time period,
utilization percentage of the surgeon,
burnout level of the surgeon,
patient panel burden of the surgeon,
experience level of an anesthesiologist,
quality of the anesthesiologist,
rating of operating room,
experience level of nurse,
patient allergies,
patient historical surgeries, or
patient comorbidities.

9. A computing system comprising:
one or more storage devices configured to store factor values for a plurality of factors that affect a risk of surgical error occurring when a surgeon performs a surgery on a patient; and
processing circuitry configured to:
for each respective factor of the plurality of factors:
obtain a factor value for the respective factor; and
apply a machine-learned weight for the respective factor to the factor value for the respective factor to determine a weighted factor value for the respective factor;
determine a risk score for the surgery based on the weighted factor values for the factors, wherein the risk score is a measure of the risk of surgical error occurring during the surgery given the factor values for the plurality of factors; and
generate, based on the risk score for the surgery, a recommendation regarding scheduling the surgery.

10. The computing system of claim 9, wherein the processing circuitry is further configured to, for at least one factor of the plurality of factors:
obtain first records of factor values of the factor for a plurality of historical surgeries and second records of whether surgical error occurred during the historical surgeries;
perform a logistic regression on the first records and the second records to determine a logistic regression curve; and
determine the weight for the factor is a coefficient that defines the logistic regression curve.

11. The computing system of claim 10, wherein, for each respective factor of the plurality of factors, the machine-learned weight for the respective factor is a respective coefficient of the logistic regression curve.

12. The computing system of claim 10, wherein the processing circuitry is further configured to:
update the first records with factor values of the factors that affect the risk of surgical error occurring when the surgeon performs the surgery on the patient; and
update the second records to indicate whether surgical error occurred when the surgeon performed the surgery on the patient.

13. The computing system of claim 9, wherein the processing circuitry is configured to, as part of obtaining the factor value for the respective factor:
obtain an initial value for the respective factor;
apply transformation logic for the respective factor to the initial value for the respective factor to determine a transformed value for the respective factor; and
apply a normalization process for the respective factor to the transformed value for the respective factor to determine the factor value for the respective value.

14. The computing system of claim 13, wherein at least one of:
the transformation logic for the respective factor is different from transformation for another factor of the plurality of factors, or
the normalization process for the respective factor is different from a normalization process for the other factor.

15. The computing system of claim 9, wherein the processing circuitry is configured to, as part of determining the risk score for the surgery based on the weighted factor values for the factors:
sum the weighted factor values for the factors to determine an initial risk score for the surgery; and
normalize the initial risk score for the surgery to determine the risk score for the surgery.

16. The computing system of claim 9, wherein the factors include one or more of:
years of experience of the surgeon,
average number of patients treated by the surgeon per time period,
average number of surgeries performed by the surgeon per time period,
utilization percentage of the surgeon,
burnout level of the surgeon,
patient panel burden of the surgeon,
experience level of an anesthesiologist,
quality of the anesthesiologist,
rating of operating room,
experience level of nurse,
patient allergies,
patient historical surgeries, or
patient comorbidities.

17. A non-transitory computer-readable storage medium having instructions stored thereon that, when executed, cause a computing system to:
for each respective factor of a plurality of factors that affect a risk of surgical error occurring when a surgeon performs a surgery on a patient:
obtain a factor value for the respective factor; and
apply a machine-learned weight for the respective factor to the factor value for the respective factor to determine a weighted factor value for the respective factor;
determine a risk score for the surgery based on the weighted factor values for the factors, wherein the risk score is a measure of the risk of surgical error occurring during the surgery given the factor values for the plurality of factors; and
generate, based on the risk score for the surgery, a recommendation regarding scheduling the surgery.

18. The non-transitory computer-readable storage medium of claim 17, wherein the instructions, when executed further cause the computing system to, for at least one factor of the plurality of factors:
obtain first records of factor values of the factor for a plurality of historical surgeries and second records of whether surgical error occurred during the historical surgeries;
perform a logistic regression on the first records and the second records to determine a logistic regression curve; and
determine the weight for the factor is a coefficient that defines the logistic regression curve.

19. The non-transitory computer-readable storage medium of claim 18, wherein, for each respective factor of the plurality of factors, the machine-learned weight for the respective factor is a respective coefficient of the logistic regression curve.

20. The non-transitory computer-readable storage medium of claim 17, wherein the instructions that cause the computing system to obtain the factor value for the respective factor comprise instructions that, when executed, cause the computing system to:
obtain an initial value for the respective factor;
apply transformation logic for the respective factor to the initial value for the respective factor to determine a transformed value for the respective factor; and
apply a normalization process for the respective factor to the transformed value for the respective factor to determine the factor value for the respective value.
